(19) United States
(12) Patent Application Publication
(10) Pub. No.: US 2006/0014730 A1
(43) Pub. Date: Jan. 19, 2006

(54) Ansamycin formulations and methods for producing and using same

(75) Inventors: Edgar H. Ulm, Encinitas, CA (US); Andrew X. Chen, San Diego, CA (US); Marcus F. Boehm, San Diego, CA (US)

Correspondence Address: WILSON SONSINI GOODRICH & ROSATI, 650 PAGE MILL ROAD, PALO ALTO, CA, 94304-1050, US

(73) Assignee: Conforma Therapeutics Corporation

(21) Appl. No.: 10/510,807

(22) PCT Filed: Apr. 4, 2003
(86) PCT No.: PCT/US03/10533

§ 371 Date: Aug. 11, 2005

Related U.S. Application Data
(60) Provisional application No. 60/371,668, filed on Apr. 10, 2002.

(51) Int. Cl. A61K 31/33

(52) U.S. Cl. 514/183

(57) ABSTRACT

Ansamycin formulations and methods of producing and using the same are described and claimed. The formulations are emulsions that can be used directly in a patient, or be lyophilized and/or frozen, to be later used, e.g., upon re- or further hydration or processing.

### RELATED APPLICATIONS

This application claims priority to Ulm et al., NOVEL ANSAMYCIN FORMULATIONS AND METHODS FOR PRODUCING AND USING SAME, U.S. Provisional Application Ser. No. 60/371,668, filed Apr. 10, 2002, which is herein incorporated by reference in its entirety including all drawings.

### FIELD OF INVENTION

The invention relates in general to pharmaceutical formulations and methods, and in more specific embodiments to emulsified formulations of ansamycins, e.g., 17-AAG.

### BACKGROUND

The following description includes information that may be useful in understanding the present invention. It is not an admission that any of the information provided herein is prior art or relevant to the presently claimed inventions, or that any publication specifically or implicitly referenced is prior art.

17-allylamino-geldanamycin (17-AAG) is a synthetic analog of geldanamycin (GDM). Both molecules belong to a broad class of antibiotic molecules known as ansamycins. GDM, as first isolated from the microorganism Streptomyces hygroscopicus, was originally identified as a potent inhibitor of certain kinases, and was later shown to act by stimulating kinase degradation, specifically by targeting “molecular chaperones,” e.g., heat shock protein 90s (HSP90s). Subsequently, various other ansamyins have demonstrated more or less such activity, with 17-AAG being among the most promising and the subject of intensive clinical studies currently being conducted by the National Cancer Institute (NCI). See, e.g., Federal Register, 66(129): 35443-35444; Erlichman et al., Proc. AACR (2001), 42, abstract 4474.

HSP90s are ubiquitous chaperone proteins that are involved in folding, activation and assembly of a wide range of proteins, including key proteins involved in signal transduction, cell cycle control and transcriptional regulation. Researchers have reported that HSP90 chaperone proteins are associated with important signaling proteins, such as steroid hormone receptors and protein kinases, including, e.g., Raf-1, EGFR, v-Src family kinases, Cdk4, and ErbB-2 (Buchner J., 1999, TIBS, 24:136-141; Stepanova, L. et al., 1996, Genes Dev. 10:1491-502; Dai, K. et al., 1996, J. Biol. Chem. 271:22030-4). Studies further indicate that certain co-chaperones, e.g., Hsp70, p60/Hop/Stil, Hip, Bag1, HSP40/Hdj2/Hsj1, immunophilins, p23, and p50, may assist HSP90 in its function (see, e.g., Caplan, A., Trends in Cell Biol., 9: 262-68 (1999).

Ansamycin antibiotics, e.g., herbimycin A (HA), geldanamycin (GM), and 17-AAG are thought to exert their anticancerous effects by tight binding of the N-terminus ATP-binding pocket of HSP90 (Stebbins, C. et al., 1997, Cell, 89:239-250). This pocket is highly conserved and has weak homology to the ATP-binding site of DNA gyrase (Stebbins, C. et al., supra; Grenert, J. P. et al., 1997, J. Biol. Chem., 272:23843-50). Further, ATP and ADP have both been shown to bind this pocket with low affinity and to have weak ATPase activity (Proromou, C. et al., 1997, Cell, 90: 65-75; Panaretou, B. et al., 1998, EMBO J, 17: 4829-36). In vitro and in vivo studies have demonstrated that occupancy of this N-terminal pocket by ansamycins and other HSP90 inhibitors alters HSP90 function and inhibits protein folding. At high concentrations, ansamycins and other HSP90 inhibitors have been shown to prevent binding of protein substrates to HSP90 (Scheibel, T., H. et al., 1999, Proc. Natl. Acad. Sci. U S A 96:1297-302; Schulte, T. W. et al., 1995, J. Biol. Chem. 270:24585-8; Whitesell, L., et al., 1994, Proc. Natl. Acad. Sci. USA 91:8324-8328). Ansamycins have also been demonstrated to inhibit the ATP-dependent release of chaperone-associated protein substrates (Schneider, C., L. et al., 1996, Proc. Natl. Acad. Sci. USA, 93:14536-41; Sepp-Lorenzino et al., 1995, J. Biol. Chem. 270:16580-16587). In either event, the substrates are degraded by a ubiquitin-dependent process in the proteasome (Schneider, C., L., supra; Sepp-Lorenzino, L., et al., 1995, J. Biol. Chem., 270:16580-16587; Whitesell, L. et al., 1994, Proc. Natl. Acad. Sci. USA, 91: 8324-8328).

This substrate destabilization occurs in tumor and non-transformed cells alike and has been shown to be especially effective on a subset of signaling regulators, e.g., Raf (Schulte, T. W. et al., 1997, Biochem. Biophys. Res. Commun. 239:655-9; Schulte, T. W., et al., 1995, J. Biol. Chem. 270:24585-8), nuclear steroid receptors (Segnitz, B., and U. Gehring. 1997, J. Biol. Chem. 272:18694-18701; Smith, D. F. et al., 1995, Mol. Cell. Biol. 15:6804-12), v-src (Whitesell, L., et al., 1994, Proc. Natl. Acad. Sci. USA 91:8324-8328) and certain transmembrane tyrosine kinases (Sepp-Lorenzino, L. et al.,. 1995, J. Biol. Chem. 270:16580-16587) such as EGF receptor (EGFR) and Her2/Neu (Hartmann, F., et al., 1997, Int. J. Cancer 70:221-9; Miller, P. et al., 1994, Cancer Res. 54:2724-2730; Mimnaugh, E. G., et al., 1996, J. Biol. Chem. 271:22796-801; Schnur, R. et al., 1995, J. Med. Chem. 38:3806-3812), CDK4, and mutant p53. Erlichman et al., Proc. AACR (2001), 42, abstract 4474. The ansamycin-induced loss of these proteins leads to the selective disruption of certain regulatory pathways and results in growth arrest at specific phases of the cell cycle (Muise-Heimericks, R. C. et al., 1998, J. Biol. Chem. 273:29864-72), and apoptsosis, and/or differentiation of cells so treated (Vasilevskaya, A. et al., 1999, Cancer Res., 59:3935-40).

Recently, Nicchitta et al., WO 01/72779 (PCT/US01/09512), demonstrated that HSP90 can assume a different conformation upon heat shock and/or binding by the fluorophore bis-ANS. Specifically, Nicchitta et al. demonstrated that this induced conformation exhibits a higher affinity for certain HSP90 ligands than for a different form of HSP90 that predominates in normal cells. Commonly-owned application PCT/US02/39993 carries this discovery even further by demonstrating the utility and uses of cancer cell lystates as excellent sources of high affinity HSP90.

In addition to anti-cancer and antitumorgenic activity, HSP90 inhibitors have also been implicated in a wide variety of other utilities, including use as anti-inflammation agents, anti-infectious disease agents, agents for treating autoimmunity, agents for treating stroke, ischemia, cardiac disorders and agents useful in promoting nerve regeneration (See, e.g., Rosen et al., WO 02/09696 (PCT/US01/23640); Degranco et al., WO 99/51223 (PCT/US99/07242); Gold, U.S. Pat. No. 6,210,974 B1; DeFranco et al., U.S. Pat. No. 6,174,875). Overlapping somewhat with the above, there are reports in the literature that fibrogenetic disorders including but not limited to scleroderma, polymyositis, systemic lupus, rheumatoid arthritis, liver cirrhosis, keloid formation, interstitial nephritis, and pulmonary fibrosis also may be treatable. (Strehlow, WO 02/02123; PCT/US01/20578). Still further HSP90 modulation, modulators and uses thereof are reported in PCT/US03/04283, PCT/US02/35938, PCT/US02/16287, PCT/US02/06518, PCT/US98/09805, PCT/US00/09512, PCT/US01/09512, PCT/US01/23640, PCT/US01/46303, PCT/US01/46304, PCT/US02/06518, PCT/US02/29715, PCT/US02/35069, PCT/US02/35938, PCT/US02/39993, 60/293,246, 60/371,668, 60/331,893, 60/335,391, 06/128,593, 60/337,919, 60/340,762, and 60/359,484.

At present, ansamycins like many other lipophilic drugs are difficult to prepare for pharmaceutical applications, especially injectable intravenous formulations. To date, attempts have been made to use lipid vesicles and oil-in-water type emulsions, but these have thus far required complicated processing steps, harsh or clinically unacceptable solvents, and/or resulted in formulation instability. See generally Vemuri, S. and Rhodes, C. T., Preparation and characterization of liposomes as therapeutic delivery systems: a review, Pharmaceutica Acta Helvetiae 70, pp. 95-111 (1995); see also PCT/US99/30631, published Jun. 29, 2000 as WO 00/37050.

A need exists for alternative formulation methods and products that improve one or more of these deficiencies.

### SUMMARY OF THE INVENTION

The invention features novel pharmaceutical formulations and methods of preparing and using the same. In a first aspect, the invention features a method comprising the steps: (a) providing a drug dissolved in ethanol; (b) mixing the product of step (a) with a medium chain triglyceride and lecithin to form a first mixture; (c) substantially removing the ethanol; (d) combining the product of step (c) with a stabilizer to form a second mixture; and (e) emulsifying the second mixture. The emulsified second mixture can be conveniently filter-sterilized and/or otherwise subjected to additional filtering steps, e.g., to reduce, or select for, emulsified droplet size or size range. The emulsified mixture can also be lyophilized and later rehydrated at will in a suitable aqueous solution for administration to a subject, e.g., intravenously.

In a second aspect, the method is not dependent on ethanol to dissolve the drug. Steps (a) and (b) of the first aspect are effectively combined into one step, with ethanol substantially absent, and hence the need to remove the ethanol as reflected in step (c) of the first aspect also eliminated. In the second aspect, the drug is brought into an oil phase solution by adding it to a preformed emulsifying agent/medium chain triglyceride solution, e.g., Phospholipon in Miglyol. The solution can be preheated and/or heated upon introduction of the drug. Temperatures in the range of 40-80° C. have been found to be particularly useful. The heated mixture may be vortexed and/or sonicated to insure desired dissolution. In such aspect, the drug preferably has a low melting point, e.g., about 175° C. or lower.

In another aspect, the invention features a method of preparing an emulsion, comprising: (a) dissolving a drug, e.g., an ansamycin, in a preformed solution comprising an emulsifying agent dissolved in a medium chain triglyceride solution, (b) combining the product of step (a) with a stabilizer, (c) emulsifying the product of step (b), (d) optionally lyophilizing the product of step (c); and (e) optionally hydrating the product of step (d).

The following embodiments may apply, as appropriate, to any given aspect of the invention.

In some embodiments, the drug is a lipophilic drug, e.g., an ansamycin such as 17-AAG (CNF-101).

In some embodiments, the medium chain triglyceride is a Miglyol®, e.g., Miglyol® 812.

In some embodiments, the medium chain triglyceride contains one or more of caprylic acid and capric acid, preferably in individual ranges of 20-80%.

In some embodiments, the emulsifying agent is or contains a phospholipid, preferably soy phosphotidylcholine, e.g., Phospholipon 90G.

In some embodiments, the preferred emulsification process includes one or more of mechanical mixing, ultrasonic irradiation, passage through a microfluidizer, and forced pressure, e.g., across a porous membrane of suitable size.

In some embodiments, the stabilizer is a bulking agent, e.g., sucrose, which can aid in stabilizing against the rigors of freeze-drying or storage at subzero temperatures.

In some embodiments, the emulsified mixture has average droplet diameter sizes of about 400 nm or less, preferably about 200 nm or less, and can be accomplished by one or more passages across one or more filters. In some embodiments, the invention contemplates serial passage across one or more filter membranes. The membranes can be of the same or different pore diameter size and have a variety of pore diameter ranges, e.g., 1-500 microns. In one embodiment, for example, passage is made across a 0.4 micron pore diameter filter, followed by passage across a 0.2 micron pore diameter filter, the latter of which may also serve the function of filter-sterilization.

In some embodiments, filter-sterilization, e.g., across a 0.2 micorn filter, is an obligatory step.

In some embodiments, the emulsified mixture has a drug concentration of about 3 mg/ml or less. By “about 3 mg/ml” is meant a numerical value of between 2.7 and 3.3 mg/ml.

In some embodiments, preparation of the formulation takes place under reduced lighting.

In some embodiments, the formulation, whether lyophilized or hydrated, is packaged in a light-resistant container or package, e.g., an ampoule or vial.

In some embodiments, the ansamycin is selected from one or more of the following: 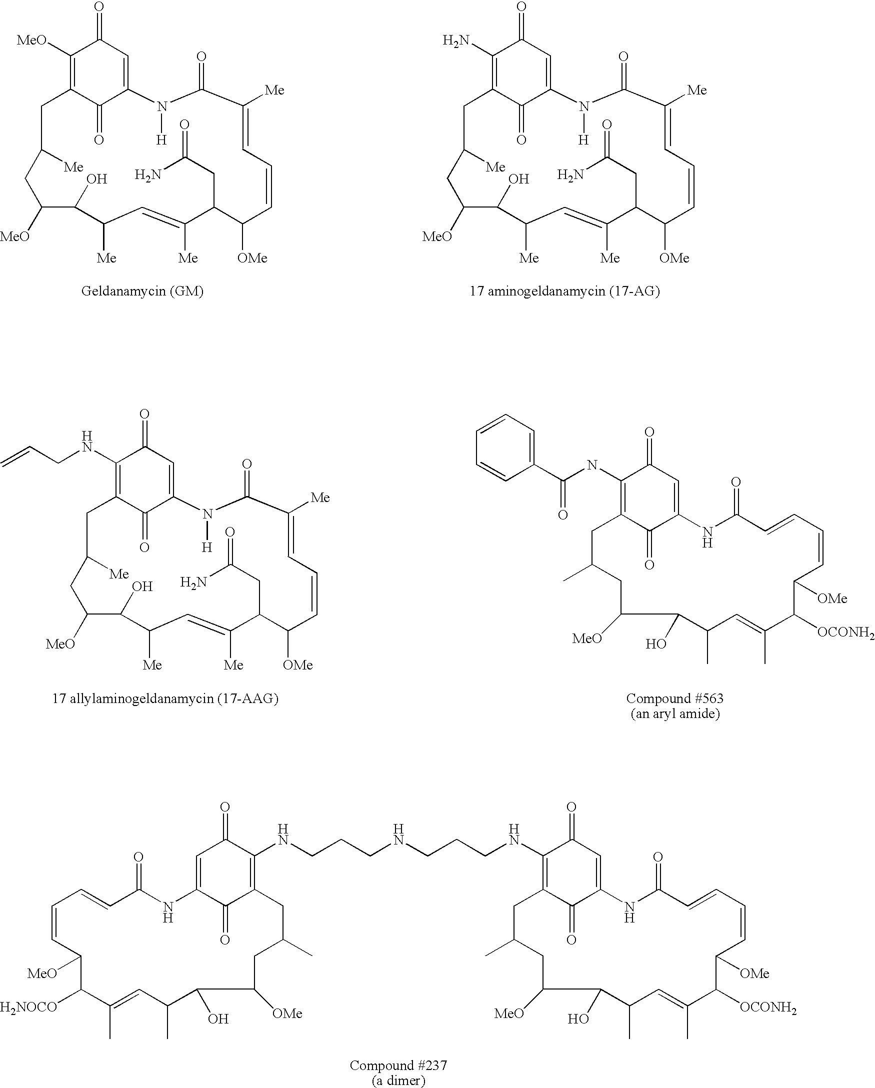
A host of other ansamycins usable according to the methods and emulsions of the invention are described in PCT/US03/04283 and other patents and patent applications described in the background of this application or otherwise known in the art.

In some embodiments, the ansamycin(s) is/are present in the form of pharmaceutically acceptable salts. One or more pharmaceutically acceptable excipients may also be present, e.g., mannitol, sucrose, and/or dextrose, various buffering agents such as sodium acetate, phosphate, lactate, tartrate and/or maleate, amino acids, sugar acids (e.g., glucocoronate and/or gluconate), and thixotropic agents such as polyethylene glycol, polyvinyl pyrrolidone and/or poloxamers (co-polymers). One particularly preferred embodiment features about 1-1.5% 237-mesylate base (w/v), further comprising about 5% mannitol, about 10-20 mM sodium acetate (x3H₂O) (pH˜5), and sterile water. This particular formulation can be adjusted to achieve any range of concentration, e.g., about 1 to about 10 mg/ml inclusive of ansamycin. pH can be manipulated using suitable acids and bases, e.g., hydrochloric acid and sodium hydroxide for adjusting sodium acetate buffered compositions. In addition or alternative to the use of sodium acetate, other buffers can be used, e.g., histidine (NMT 5 mM; pH˜5), lactic acid (˜10-20 mM; pH˜4), valine (˜10-50 mM; pH˜3), etc.

In another aspect, the invention features methods of using the pharmaceutical compositions, formulations, or products described above for treating or preventing a disorder in an organism, e.g., a mammal, by administering to the organism a pharmaceutically effective amount of product. The disorder, at least in the instance of mammalian treatment, is preferably selected from the group of disorders consisting of ischemia, proliferative disorders and neural damage. Proliferative disorders include but are not limited to tumors and cancers, inflammatory diseases, fungal infections, yeast infections, and viral infections. In some preferred embodiments, the mammal is human. In some preferred embodiments, the administration mode is intravenous, although as described in more detail, below, other modes of administration are also contemplated. Advantages of the invention include one or more of ease of manufacture, the use of clinically acceptable reagents (e.g., having reduced environmental and/or patient toxicity), enhanced formulation stability, uncomplicated shipment and warehousing, and simple pharmacy and bed-side handling. Other advantages, aspects, and embodiments will be apparent from the figures, the detailed description, and claims to follow.

Any of the above aspects and embodiments, and as further described in the claims to follow, can be combined as appropriate to achieve a suitable objective within the spirit of the invention.

### DETAILED DESCRIPTION OF THE INVENTION

The formulations of the invention have particular merit in rendering water-insoluble drugs suitable for intravenous and other types of administration to a patient. The method of formulation is relatively simple, typically utilizes clinically acceptable reagents, and results in a product that affords storage and stability advantages over existing methods and products.

In some embodiments, the method features the creation of lyophilized cakes of emulsion containing the pharmaceutical compound of interest, i.e., drug, which lyophilized form can be used for storage and prolonged stability and readily rehydrated upon demand for administration to a patient, intravenously or otherwise.

One method includes providing a pharmaceutical compound dissolved in ethanol or equivalent solvent, mixing with a medium chain fatty acid solution, substantially evaporating away the ethanol or equivalent solvent, adding emulsifying and/or bulking agents, and emulsifying. This may be done with the aid of microchannel filters through which the emulsion is passed under high pressure to select for a given diameter of emulsified product.

Another method is performed in the absence of ethanol, and includes adding drug to a preformed emulsifying agent/medium chain triglyceride solution, and with the help of heat and/or sonication adding and dissolving the drug therein.

The foregoing methods may be followed by lyophilization and rehydration at a suitable point in time. Lyophilization results in a product that is relatively stable and convenient for storage, shipping, and handling. Upon hydration and adjustment to a suitable concentration, administration may be conveniently made to a patient, intravenously or otherwise. The invention is illustrated using the ansamycin 17-AAG (CNF-101). However, it will be appreciated that the novel methods of drug formulation described herein may be applied to many other ansamycin drugs and compounds besides ansamycins, especially those having a high solubility in ethanol as opposed to water.

The term “drug” means any compound that exerts, directly or indirectly, a biological effect, in vitro or in vivo when administered to cultured cells or to an organism. The drug should be capable of encasement in liposomes and/or emulsification, and will typically, although not necessarily, be lipophilic.

The term “dissolving an ansamycin in ethanol” or “providing an ansamycin dissolved in ethanol” does not exclude the possibility that the ethanol is itself part of an aqueous solution containing some water or other solvent or solute. It further does not necessarily imply saturating the ethanol with dissolved drug, although it can.

The term “substantially removing said ethanol” means eliminating all, most, or a majority fraction of the ethanol present in the first mixture. Preferably 90% or more of the ethanol is removed. This may be accomplished by one or more means, e.g., of vacuum, flow of inert gas, decanting, and heat application, and will typically involve evaporation. In combination with increased vacuum, cooler temperatures may also be used to evaporate the ethanol.

The terms “evaporating” and “lyophilizing” do not necessarily imply 100% elimination of solvent and solution, and may entail lesser percentages of removal. Substantial removal is preferred, preferably about 95% removal.

An “inert atmospheric condition” is one that is relatively less reactive than the air of standard atmospheric conditions. The use of pure or substantially pure nitrogen gas is one example of such an inert atmospheric condition. Persons of ordinary skill in the art are familiar with others.

The term “hydrating” or “rehydrating” means adding an aqueous solution, e.g., water or a physiologically compatible buffer such as Hanks's solution, Ringer's solution, or physiological saline buffer.

The term “stabilizer” can be synonymous with “bulking agent” or “freeze-drying agent” and vice versa, although need not be.

“Pharmaceutically acceptable salts” include those derived from pharmaceutically acceptable inorganic and organic acids and bases. Examples of suitable acids include hydrochloric, hydrobromic, sulfuric, nitric, perchloric, fumaric, maleic, phosphoric, glycolic, gluconic, lactic, salicylic, succinic, toluene-p-sulfonic, tartaric, acetic, citric, methanesulfonic, formic, benzoic, malonic, naphthalene-2-sulfonic, benzenesulfonic, 1,2 ethanesulfonic acid (edisylate), galactosyl-d-gluconic acid, and the like. Other acids, such as oxalic acid, while not themselves pharmaceutically acceptable, may be employed in the preparation of salts useful as intermediates in obtaining the compounds of this invention and their pharmaceutically acceptable acid addition salts. Salts derived from appropriate bases include alkali metal (e.g., sodium), alkaline earth metal (e.g., magnesium), ammonium and N-(C.sub.1-C.sub.4 alkyl).sub.4.sup.+salts, and the like. Illustrative examples of some of these include sodium hydroxide, potassium hydroxide, choline hydroxide, sodium carbonate, and the like. Where the claims recite “a compound (e.g., compound ‘x’) or pharmaceutically acceptable salt thereof,” and only the compound is displayed, those claims are to be interpreted as embracing, in the alternative or conjunctive, a pharmaceutically acceptable salt or salts of such compound.

A “physiologically acceptable carrier” refers to a carrier or diluent that does not cause significant irritation to an organism and does not abrogate the biological activity and properties of the administered compound.

An “excipient” refers to an inert substance added to a pharmacological composition to further facilitate administration of a compound. Examples of excipients include but are not limited to calcium carbonate, calcium phosphate, various sugars and types of starch, cellulose derivatives, gelatin, vegetable oils and polyethylene glycols.

The term “about” is meant to embrace deviations of 20% from what is stated. The term “inclusive” when used in conjunction with the term “between” or “between about” means including the stated range's endpoints.

Ansamycins and Benzoquinone Ansamycins

The term “ansamycin” is a broad term including the rifamycin group of antibiotics, which are characterized by a natural ansa structure (chromophoric naphthohydro quinone group spanned by a long aliphatic bridge). Into this broad class is subsumed the sub-class, benzoquinone ansamycins. A “benzoquinone ansamycinin” as used in the claims includes any benzoquinone ansamycinin known in the art including geldanamycin, dihydrogeldanamycin, herbamycin, macbecin, etc. Ansamycins and benzoquinone ansamycins according to the invention may be synthetic, naturally-occurring, or a combination of the two, i.e., “semi-synthetic”, and may include dimers and conjugated variant and prodrug froms. Some exemplary benzoquinone ansamycins useful in the processes of the invention and their methods of preparation include but are not limited to those described, e.g., in U.S. Pat. No. 3,595,955 (describing the preparation of geldanamycin), U.S. Pat. Nos. 4,261,989, 5,387,584, and 5,932,566. Geldanamycin is also commercially available, e.g., from CN Biosciences, an Affiliate of Merck KGaA, Darmstadt, Germany, headquartered in San Diego, Calif., USA (cat. no. 345805). The biochemical purification of the geldnamycin derivative, 4,5-Dihydrogeldanamycin and its hydroquinone from cultures of Streptomyces hygroscopicus (ATCC 55256) are described in International Application Number PCT/US92/10189, assigned to Pfizer Inc., published as WO 93/14215 on Jul. 22, 1993, and listing Cullen et al. as inventors; an alternative method of synthesis for 4,5-Dihydrogeldanamycin by catalytic hydrogenation of geldanamycin is also known. See e.g., Progress in the Chemistry of Organic Natural Products, Chemistry of the Ansamycin Antibiotics, 33:278 (1976).

Solvent Addition and Removal

Solubility in ethanol depends on the properties of the compound(s) being dissolved therein, and also on other variables such as temperature and pressure. Ethanol has a boiling point of approximately 78.5° C. at sea level atmospheric conditions. The inclusion of water in ethanol raises the boiling point of the solution.

Evaporation is preferably, although not necessarily, carried out under reduced pressure, i.e., vacuum, and may be performed at any reasonable temperature and pressure, including at room temperature with a stream of nitrogen, as long as the procedure is suitable to preserve the functional integrity of the pharmaceutical agent. Commercially available rotary evaporation devices exist to accomplish solvent removal. Other devices and methods are known to the skilled artisan.

Crystallization

Crystallization is the deposition of crystals from a solution or melt of a given material. During the process of crystal formation, like molecules tend to become attached to a growing crystal composed of the same type of molecules because of a better fit in a crystal lattice for molecules of the same structure than for other molecules. If the crystallization process is allowed to occur under near equilibrium conditions, the preference of molecules to deposit on surfaces composed of like molecules will lead to an increase in the purity of the crystalline material. Thus the process of crystallization is an important method of purification. Crystallization is subsumed under the broader term “precipitation,” which indicates a dissolved compound ceasing to be dissolved, and effectively taking solid form outside of solution, whether “crystallized” or not. The precipitation or crystallization may occur over a prolonged period of time, e.g., seconds to hours or days. These processes also depend on temperature, and further depend on the properties of the specific solvent and solutes being used, with a solubility differential across warm versus cooler temperatures.

The ability to generate low melting point isoforms of lipophilic compounds such as 17-AAG is described and claimed in commonly-owned U.S. application Ser No. 60/331,893, filed Nov. 21, 2001. The differences in melting points, as exemplified with 17-AAG (exhibiting melting points of circa. 155° C. and 207° C.), can be exploited more or less successfully in connection with the drug dissolution steps claimed herein, with the lower melting point isoforms preferred in some embodiments of the instant invention. The lower melting point isoform of 17-AAG is achieved using an isopropanol crystalization rather than an ethanol-based crystalization/precipitation. Using this technique, melting points as low as 153° C. and as high as 212° C. have been measured for 17-AAG, with high purity levels essentially maintained.

Emulsions

Emulsions comprising an oil phase and an aqueous phase are widely known in the art as carriers of therapeutically active ingredients or as sources of parenteral nutrition. Emulsions can exist as either oil-in-water or water-in-oil forms. If, as is the case in the current instance, the therapeutic ingredient is particularly soluble in the oil phase the oil-in-water type is the preferred embodiment. Simple emulsions are thermodynamically unstable systems from which the oil and aqueous phases separate (coalescence of oil droplets). Incorporation of an emulsifying agent(s) into the emulsion is critical to reduce the process of coalescence to insignificant levels.

Oils

Although any nontoxic oil/lipid may be used with the invention, preferred are mono-, di- and triglycerides, especially triglycerides, and most preferably medium chain triglycerides. “Medium chain triglycerides” are triglyceride compositions wherein the predominant constituent is a species having fatty acids of range 7-11 carbon atoms in length, and more preferably 8-10 carbon atoms in length. The fatty acids are preferably saturated, although need not be. Larger and smaller length triglycerides may be present, but are typically present in lesser abundance relative to the medium chain species. The individual triglycerides may be natural, synthetic, semi-synthetic, charged, neutral, homogenous or heterogenous with respect to the identity of the individual triglyceride molecules. The invention is illustrated using Miglyol® 812, offered by CONDEA (Cranford, N.J., USA) as an exemplary embodiment. Miglyol® 812 contains roughly 50-65% Caprylic acid (8 carbons) and 30-45% Capric acid (10 carbons). Caproic acid (6 carbon atoms) is also present, up to a maximum of about 2%, as is Lauric Acid (12 carbons). Present in still a lesser amount (1% max) is Myristic acid (14 carbons). Condea also offers Mygliol® 810, 818, 829, and 840, and it is anticipated that one or more of these other Mygliol® solutions, as well as other medium chain triglyceride solutions can also be used more or less successfully in connection with various aspects and embodiments of the invention. As to the latter, one of ordinary skill in the art knows their identity, source and/or manner of preparation, and can acquire or prepare them without undue investigation or experimentation.

To prevent or minimize oxidative degradation or lipid peroxidation, antioxidants, e.g., alpha-tocopherol and butylated hydroxytoluene, may be included in addition to, or as an alternative to, oxygen deprivation (e.g., formulation in the presence of inert gases such as nitrogen and argon, and/or the use of light resistant containers.

Emulsifying Agents

Preferred emusifying agents are lecithins, which are naturally occurring mixtures of diglycerides of stearic, palmitic, and oleic acids, linked to the choline ester of phosphoric acid. Other emulsifying agents may include various surfactants (e.g. anionic, cationic and nonionic surfactants). The surfactant/emulsifying agent may be a phospholipid, e.g., an egg or vegetable oil phospholipid or phosphatidylcholine. Preferred for use in the methods of the invention is soy phospholecithin, e.g., Phopholipon 90G as supplied by American Lecithen Company (Oxford, Conn., USA).

The surfactant/emulsifying agent is typically present in a concentration of about 0.5-25% w/v based on the amount of the water and/or other components into which the surfactant is dissolved. Preferably, the surfactant is present in a concentration of about 0.5-10% w/v, most preferably about 1-8% w/v.

Examples of anionic surfactants include sodium lauryl sulfate, lauryl sulfate triethanolamine, sodium polyoxyethylene lauryl ether sulfate, sodium polyoxyethylene nonylphenyl ether sulfate, polyoxyethylene lauryl ether sulfate triethanolamine, sodium cocoylsarcosine, sodium N-cocoylmethyltaurine, sodium polyoxyethylene (coconut)alkyl ether sulfate, sodium diether hexylsulfosuccinate, sodium a-olefin sulfonate, sodium lauryl phosphate, sodium polyoxyethylene lauryl ether phosphate, perfluoroalkyl carboxylate salt (manufactured by Daikin Industries Ltd. under the trade name of UNIDINE DS-101 and 102).

Examples of cationic surfactants include dialkyl(C₁₂-C₂₂)dimethylammonium chloride, alkyl(coconut)dimethylbenzylammonium chloride, octadecylamine acetate salt, tetradecylamine acetate salt, tallow alkylpropylenediamine acetate salt, octadecyltrimethylammonium chloride, alkyl(tallow) trimethylammonium chloride, dodecyltrimethylammonium chloride, alkyl(coconut) trimethylammonium chloride, hexadecyltrimethylammonium chloride, biphenyltrimethylammonium chloride, alkyl(tallow)-imidazoline quaternary salt, tetradecylmethylbenzylammonium chloride, octadecyidimethylbenzylammonium chloride, dioleyidimethylammonium chloride, polyoxyethylene dodecylmonomethylammonium chloride, polyoxyethylene alkyl(C₁₂-C₂₂)benzylammonium chloride, polyoxyethylene laurylmonomethyl ammonium chloride, 1- hydroxyethyl-2-alkyl(tallow)-imidazoline quaternary salt, and a silicone cationic surfactant having a siloxane group as a hydrophobic group, a fluorine-containing cationic surfactant having a fluoroalkyl group as a hydrophobic group (manufactured by Daikin Industries Ltd. under the trade name of UNIDINE DS-202).

Examples of nonionic surfactants include polyoxyethylene lauryl ether, polyoxyethylene tridecyl ether, polyoxyethylene cetyl ether, polyoxyethylene polyoxypropylene cetyl ether, polyoxyethylene stearyl ether, polyoxyethylene oleyl ether, polyoxyethylene nonylphenyl ether, polyoxyethylene octylphenyl ether, polyoxyethylene monolaurate, polyoxyethylene monostearate, polyoxyethylene monooleate, sorbitan monolaurate, sorbitan monostearate, sorbitan monopalmitate, sorbitan monostearate, sorbitan monooleate, sorbitan sesquioleate, sorbitan trioleate, polyoxyethylene sorbitan monolaurate, polyoxyethylene sorbitan monopalmitate, polyoxyethylene sorbitan monostearate, polyoxyethylene sorbitan monooleate, polyoxyethylene polyoxypropylene block polymer, polyglycerin fatty acid ester, polyether-modified silicone oil (manufactured by Toray Dow Corning Silicone Co., Ltd. under the trade names of SH3746, SH3748, SH3749 and SH3771), perfluoroalkyl ethyleneoxide adduct (manufactured by Daikin Industries Ltd. under the trade names of UNIDINE DS-401 and DS-403), fluoroalkyl ethyleneoxide adduct (manufactured by Daikin Industries Ltd. under the trade name of UNIDINE DS-406), and perfluoroalkyl oligomer (manufactured by Daikin Industries Ltd. under the trade name of UNIDINE DS-451).

Stabilizers/Bulking Agents

Further falling within the definition of excipient are bulking agents. A bulking agent generally provides mechanical support for a lyophile formulation by allowing the dry formulation matrix to maintain its conformation. Preferred are sugars. Sugars as used herein include but are not limited to monosaccharides, disaccharides, oligosaccharides and polysaccharides. Specific examples include but are not limited to fructose, glucose, mannose, trehalose, sorbose, xylose, maltose, lactose, sucrose, dextrose, and dextran. Sugar also includes sugar alcohols, such as mannitol, sorbitol, inositol, dulcitol, xylitol and arabitol. Mixtures of sugars may also be used in accordance with this invention. Various bulking agents, e.g., glycerol, sugars, sugar alcohols, and mono and disaccharides may also serve the function of isotonizing agents.

Bulking agents for use with the invention are limited only by chemico-physical considerations, such as solubility, ability to preserve the droplet size and emulsion integrity during freezing, drying, stogage and rehydration and lack of reactivity with the active drug/compound, and limited as well by route of administration. It is preferred that the bulking agents be chemically inert to drug(s) and have no or extremely limited detrimental side effects or toxicity under the conditions of use. In addition to bulking agent carriers, other carriers that may or may not serve the purpose of bulking agents include, e.g., adjuvants, excipients, and diluents as well known and readily available in the art.

A preferred bulking agent for the invention is sucrose. Without being bound by theory, sucrose is thought to form an amorphous glass upon freezing and subsequent lyophilization, allowing for potential stability enhancement of the formulation by forming a dispersion of the oil droplets containing the active ingredient in a rigid glass. Stability may also be enhanced by virtue of the sugar acting as a replacement for the water lost upon lyophilization. The sugar molecules, rather than the water molecules, become bonded to the interfacial phospholipid through hydrogen bonds. Other bulking agents which possess these characteristics and which may be substituted include but are not limited to cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may also be added, e.g., cross-linked polyvinyl pyrrolidone, agar, or alginic acid or a salt thereof such as sodium alginate.

Emulsification

Emulsification can be effected by a variety of well known techniques, e.g., mechanical mixing, homogenization (e.g., using a polytron or Gaulin high-energy-type instrument), vortexing, and sonication. Sonication can be effected using a bath-type or probe-type instrument. Microfluidizers are commercially available, e.g., from Microfluidics Corp., Newton, Mass., are further described in U.S. Pat. No. 4,533,254, and make use of pressure-assisted passage across narrow orifices, e.g., as contained in various commercially available polycarbonate membranes. Low pressure devices also exist that can be used. These high and low pressure devices can be used to select for and/or modulate vesicle size.

Sterilization by filtration techniques. Filtration can include a pre-filtration through a larger diameter filter, e.g., a 0.45 micron filter, and then through smaller filter, e.g., a 0.2 micron filter. The preferred filter medium is cellulose acetate (Sartorius-Sartobran™).

Lyophilization

Lyophilization is the removal or substantial removal of liquid from a sample, e.g., by sublimation, and as described in the section above entitled “solvent removal.”

Characterization and Assessment of Chemical and Physical Stability

Phospholipids and degradation products may be determined after being extracted from emulsions. The lipid mixture can then be separated in a two-dimensional thin-layer chromatographic (TLC) system or in a high performance liquid chromatographic (HPLC) system. In TLC, spots corresponding to single constituents can be removed and assayed for phosphorus. Total phosphorous in a sample can be quantitatively determined, e.g., by a procedure using a spectrophotometer to measure the intensity of blue color developed at 825 nm against water. In HPLC, phosphatidylcholine (PC) and phosphotidylglycerol (PG) can be separated and quantified with accuracy and precision. Lipids can be detected in the region of 203-205 nm. Unsaturated fatty acids exhibit high absorbance while the saturated fatty acids exhibit lower absorbance in the 200 nm wavelength region of the UV spectrum. As an example, Vemuri and Rhodes, supra, described the separation of egg yolk PC and PG on Licrosorb Diol and Licrosorb S1-60. The separations used a mobile phase of acetonitrile-methanol with 1% hexane-water (74:16:10 v/v/v). In 8 minutes, separation of PG from PC was observed. Retention times were approximately 1.1 and 3.2 min, respectively.

Emulsion visual appearance, average droplet size, and size distribution are important parameters to observe and maintain. There are a number of methods to evaluate these parameters. For example, dynamic light scattering and electron microscopy are two techniques that can be used. See, e.g., Szoka and Papahadjopoulos, Annu. Rev. Biophys. Bioeng., 9:467-508 (1980). Morphological characterization, in particular, can be accomplished using freeze fracture electron microscopy. Less powerful light microscopes can also be used.

Emulsion droplet size distribution can be determined, e.g., using a particle size distribution analyzer such as the CAPA-500 made by Horiba Limited (Ann Arbor, Mich., USA), a Coulter counter (Beckman Coulter Inc., Brea, Calif., USA), or a Zetasizer (Malvem Instruments, Southborough, Mass., USA).

Stability Determination Using HPLC

Similar to the methods described above for the lipid components of the emulsion, the chemical stability of the therapeutically active ingredient, e.g, 17-AAG, can be assessed by HPLC after extraction of the emulsion. Specific assay procedures can be developed that allow for the separation of the therapeutically active ansamycin from its degradation products. The extent of degradation can be assessed either from the decrease in signal in the HPLC peak associated with the therapeutically active ansamycins and/or by the increase in signal in the HPLC peak(s) associated with degradation products. Ansamycins, relative to other components of the emulsion components, are easily and quite specifically detected at their absorbance maximum of 345 nm.

Modes of Formulation and Administration

Although intravenous administration is preferred in various aspects and embodiments of the invention, one of ordinary skill will appreciate that the methods can be modified or readily adapted to accommodate other administration modes, e.g., oral, aerosol, parenteral, subcutaneous, intramuscular, intraperitoneal, rectal, vaginal, intratumoral, or peritumoral. The following discussion is largely known to the person of skill but is nevertheless provided as a backdrop to illustrate other possibilities for the invention. It will be appreciated that various of the commentary below overlaps with what has already been discussed above.

Pharmaceutical compositions may be manufactured utilizing conventional mixing, dissolving, granulating, dragee-making, levigating, emulsifying, encapsulating, entrapping or lyophilizing processes.

Pharmaceutically acceptable compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Some excipients and their use in the preparation of formulations have already been described. Others are known in the art, e.g., as described in PCT/US99/30631, Remmington's Pharmaceutical Sciences, Meade Publishing Co., Easton, Pa. (most recent edition), and Goodman and Gilman's The Pharmaceutical Basis of Therapeutics, Pergamon Press, New York, N.Y. (most recent edition).

For injection, the agents may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

Formulations of the invention, as described previously, and upon hydration of the lyophilized cakes, are well suited for immediate or near-immediate parenteral administration by injection, e.g., by bolus injection or continuous infusion. Formulations for injection may be presented in unit dosage form, e.g., in ampoules or in multi-dose containers, with an added preservative. As discussed previously, lyophilized products are a particularly preferred embodiment for the invention and ampoules or other packaging, optionally light-resistant, may contain this lyophilized product, which may then be conveniently (re)hydrated prior to administration to a patient.

Dose Range

A phase I pharmacologic study of 17-AAG in adult patients with advanced solid tumors determined a maximum tolerated dose (MTD) of 40 mg/m² when administered daily by 1-hour infusion for 5 days every three weeks. Wilson et al., Am. Soc. Clin. Oncol., abstract, Phase I Pharmacologic Study of 17-(Allylamino)-17-Demethoxygeldanamycin(AAG) in Adult Patients with Advanced Solid Tumors (2001). In this study, mean+/−SD values for terminal half-life, clearance and steady-state volume were determined to be 2.5+/−0.5 hours, 41.0+/−13.5 L/hour, and 86.6+/−34.6 L/m². Plasma Cmax levels were determined to be 1860+/−660 nM and 3170+/−1310 nM at 40 and 56 mg/m2. Using these values as guidance, it is anticipated that the range of useful patient dosages for formulations of the present invention will include between about 0.40 mg/m² and 4000 mg/m² of active ingredient. M² represents surface area. Standard algorithms exist to convert mg/m² to mg drug/kg bodyweight.

The following Examples are offered by way of illustration only, and components and steps included therein are not intended to be limiting of the invention unless specifically recited in the claims. Examples 1-4 are borrowed from commonly owned provisional application Ser. No. 60/326,639, entitled Process for Preparing 17-Allyl Amino Geldanamycin (17-AAG) and Other Ansamycins, filed Sep. 24, 2001, and commonly owned provisional application Ser. No. 60/331,893, filed Nov. 21, 2001.

### EXAMPLE 1

### Preparation of 17-AAG

To 45.0 g (80.4 mmol) of geldanamycin in 1.45 L of dry THF in a dry 2 L flask was added drop-wise over 30 minutes, 36.0 mL (470 mmol) of allyl amine in 50 mL of dry THF. The reaction mixture was stirred at room temperature under nitrogen for 4 hr at which time TLC analysis indicated the reaction was complete [(GDM: bright yellow: R=0.40; (5% MeOH-95% CHCl₃); 17-AAG: purple: Rf-0.42 (5% MeOH-95% CHCl₃)]. The solvent was removed by rotary evaporation and the crude material was slurried in 420 mL of H₂O:EtOH (90:10) at 25° C., filtered and dried at 45° C. for 8 hr to give 40.9 g (66.4 mmol) of 17-AAG as purple crystals (82.6% yield, >98% pure by HPLC monitored at 254 nm). MP 206-212° C. 1H NMR and HPLC are consistent with the desired product.

### EXAMPLE 2

### Preparation of 17-AAG

An alternative method of purification is to dissolve the crude 17-AAG from example 1 in 800 mL of 2-propyl alcohol (isopropanol) at 80° C. and then cool to room temperature. Filtration followed by drying at 45° C. for 8 hr gives 44.6 g (72.36 mmol) of 17-AAG as purple crystals (90% yield, >99% pure by HPLC monitored at 254 nm). MP 147-153° C. 1H NMR and HPLC are consistent with the desired product.

### EXAMPLE 3

### Preparation of 17-AAG

An alternative method of purification is to slurry the 17-AAG product from example 2 in 400 mL of H₂O:EtOH (90:10) at 25° C., filtered and dried at 45° C. for 8 hr to give 42.4 g (68.6 mmol) of 17-AAG as purple crystals (95% yield, >99% pure by HPLC monitored at 254 nm). MP 147-153° C. ¹H NMR and HPLC are consistent with the desired product.

### EXAMPLE 4

### Preparation of a 17-AAG Emulsion

The 17-AAG obtained from any one of Examples 1-4, above, is dissolved in ethanol. The following Table illustrates a 4000 gm batch preparation of 17-AAG made according to one embodiment of the invention. The skilled artisan will recognize that the procedure can be scaled up or down, that variations can be made with respect to the amounts of individual components, etc., and that additional components not listed may also be added.

| | | Grams for |
|---|---|---|
| Component | % w/w | 4000 g batch |
| CNF-101 (17-AAG) | 0.2 | 8 |
| Miglyol 812 | 15 | 600 |
| Phospholipon 90G | 7.5 | 300 |
| EDTA disodium, dihydrate, USP | 0.005 | 0.2 |
| Sucrose, NF | 15 | 600 |
| Water-for-injection, USP | QS | QS |
| 0.2N NaOH | To adjust pH to 6.0 ± 0.2 | As needed |

17-AAG (CNF-101) is weighed in a 5 L polypropylene beaker. Ethanol is added in an amount approximately 50× the drug weight and the solution sonicated in a water bath to disperse the drug. Miglyol 812 (Sasol North America Inc; Houston, Tex., USA) and Phospholipon 90G (American Lecithen Co., Oxford, Conn., USA) are then added to the dispersion and the mixture placed on a stir plate and stirred until the solids are more or less completely dissolved. A sonicator bath and/or heat to approximately 45° C. may be used to help dissolve the solids. The solution may be checked using an optical microscope to ensure desired dissolution.

A stream of dry air or nitrogen (National Formulary) gas is forced over the liquid surface in combination with vigorous stirring to evaporate the ethanol until the ethanol content is reduced, preferably to less than 50% of its initial presence, more preferably to less than 10%, and most preferably to about 5% or less, w/w. The solution can be checked under an optical microscope equipped with polarizing filters to ensure the desired level of dissolution, preferably complete dissolution (no crystals or precipitate).

EDTA (disodium, dihydrate, USP), sucrose, and water for injection (WFI) are weighed into a 5 L polypropylene beaker and stirred until the solids are dissolved. The aqueous phase is then added to the oil phase and thorough mixing effected using a high-speed emulsifier equipped with an emulsion head at 5000 RPM until the oil adhering to the surface is “stripped off.” Shearing rate is then increased to 10000 RPM for 2-5 minutes to obtain a uniform primary emulsion. Laser light scattering (LLS) may be used to measure the average droplet size, and the solution may further be checked, e.g., under an optical microscope to determine the relative presence or absence of crystals and solids.

The emulsion pH is adjusted to 6.0+/−0.2 with 0.2N NaOH. The primary emulsion is then passed through a Model 11OS microfluidizer (Microfluidics Inc., Newton, Mass., USA) operating at static pressure of about 110 psi (operating pressure of 60-95 psi) with a 75-micron emulsion interaction chamber (F20Y) for 6-8 passages until the average droplet size is ≦190 nm. LLS may be used following the individual passages to evaluate progress. The solution may further be checked for the presence of crystals using polarized light under an optical microscope.

In a laminar flow hood, the emulsion is then passed across a 0.45 micron Gelman mini capsule filter (Pall Corp., East Hills, N.Y., USA), and then across a sterile 0.2 micron Sartorius Sartobran P capsule filter (500 cm²) (Sartorius AG, Goettingen, Germany). Pressure up to 60 PSI is used to maintain a smooth and continuous flow. Filtrate is collected in one or more polypropylene bottles and immediately placed in a −20° C. freezer. A 1-ml aliquot may be set aside for testing using laser light scattering (LLS) and/or high performance liquid chromatography (HPLC).

### EXAMPLE 5

### Alternative Preparation of 17-AAG Emulsion Formulation

When using ethanol to facilitate the dissolution of 17-AAG into the oil phase of the emulsion, it is most common to first dissolve 17-AAG in the ethanol using sonication followed by addition of the emulsifying agent and medium chain triglyceride to that solution. Sonication and stirring are then employed to effect solution of all the components.

Alternatively, 17-AAG can be brought into solution in the oil phase without ethanol being present by heating a preformed emulsifying agent in triglyceride solution, e.g., Phospholipon in Miglyol® 812, preferably to 65° C. or more, adding to this the drug, e.g., 17-AAG, and mixing, e.g., by stirring and/or sonication. It has also been discovered that a lower melting point form of 17-AAG prepared through crystallization of 17-AAG from isopropanol rather than ethanol more readily can be dissolved into the Phospholipon in Miglyol solution at room temperature. This discovery can be extrapolated to other drugs that exhibit multiple “isoforms” having different melting points.

The products of both examples 5 and 6 result in a light purple, milky emulsion having a mean oil droplet size in the range of about 200 nm or less. The droplet size is stable at −20° C., 2-8° C., or room temperature for periods in excess of two months. Concentrations as high as 3 mg/ml have been achieved overall and as high as 20-30 mg/ml in the oil phase alone. When stored at 40° C., degradants of 17-AAG are seen after approximately 2 weeks.

The mixed-solvent solution of the drug is subjected to vacuum evaporation of the ethanol component resulting in a solution of 17-AAG in Miglyol. Emulsification can be accomplished by mechanical mixing, by treating with ultrasonic irradiation, and finally by passage through a microfluidizer, although it will be understood that the terms “emulsify” and “emulsification” should not be limited to such processing events and that other emulsification techniques exist and can be used alternatively or in tandem with one or more of the preceding techniques.

### EXAMPLE 6

### Ansmaycins Other Than 17-AAG

Essentially any ansamycin can be substituted for 17-AAG and formulated as described in the above examples. Various such ansamycins and their preparation are detailed in PCT/US03/04283. The preparation of two such preferred ansamycins, compounds 563 and 237, is repeated below.

Compound 563: 17-(benzoyl)-aminogeldanamycin. A solution of 17-aminogeldanamycin (1 mmol) in EtOAc was treated with Na₂S₂O₄ (0.1 M, 300 ml) at RT. After 2 h, the aqueous layer was extracted twice with EtOAc and the combined organic layers were dried over Na₂SO₄, concentrated under reduce pressure to give 18,21-dihydro-17-aminogeldanamycin as a yellow solid. This latter was dissolved in anhydrous THF and transferred via cannula to a mixture of benzoyl chloride (1.1 mmol) and MS4 Å (1.2 g). Two hours later, EtN(i-Pr)₂ (2.5 mmol) was further added to the reaction mixture. After overnight stirring, the reaction mixture was filtered and concentrated under reduce pressure. Water was then added to the residue which was extracted with EtOAc three times, the combined organic layers were dried over Na₂SO₄ and concentrated under reduce pressure to give the crude product which was purified by flash chromatography to give 17-(benzoyl)-aminogeldanamycin. Rf=0.50 in 80:15:5 CH₂Cl₂: EtOAc: MeOH. Mp=218-220° C. 1H NMR (CDCl₃) 0.94 (t, 6H), 1.70 (br s, 2H), 1.79 (br s, 4H), 2.03 (s, 3H), 2.56 (dd, 1H), 2.64 (dd, 1H), 2.76-2.79 (m, 1H), 3.33 (br s, 7H), 3.44-3.46 (m, 1H), 4.325 (d, 1H), 5.16 (s, 1H), 5.77 (d, 1H), 5.91 (t, 1H), 6.57 (t, 1H), 6.94 (d, 1H), 7.48 (s, 1H), 7.52 (t, 2H), 7.62 (t, 1H), 7.91 (d, 2H), 8.47 (s, 1H), 8.77 (s, 1H).

Compound 237: A dimer. 3,3′-diamino-dipropylamine (1.32 g, 9.1 mmol) was added dropwise to a solution of Geldanamycin (10 g, 17.83 mmol) in DMSO (200 ml) in a flame-dried flask under N₂ and stirred at room temperature. The reaction mixture was diluted with water after 12 hours. A precipitate was formed and filtered to give the crude product. The crude product was chromatographed by silica chromatography (5% CH₃OH/CH₂Cl₂) to afford the desired dimer as a purple solid. The pure purple product was obtained after flash chromatography (silica gel); yield: 93%; mp 165° C.; 1H NMR (CDCl₃) 0.97 (d, J=6.6 Hz, 6H, 2CH3), 1.0 (d, J=6.6 Hz, 6H, 2CH3), 1.72 (m, 4H, 2 CH2), 1.78 (m, 4H, 2CH2), 1.80 (s, 6H, 2 CH3), 1.85 (m, 2H, 2CH), 2.0 (s, 6H, 2CH3), 2.4 (dd, J=11 Hz, 2H, 2CH), 2.67 (d, J=15 Hz, 2H, 2CH), 2.63 (t, J=10 HZ, 2H, 2CH), 2.78 (t, J=6.5 Hz, 4H, 2CH2), 3.26 (s, 6H, 2OCH3), 3.38 (s, 6H, 2OCH3), 3.40 (m, 2H, 2CH), 3.60 (m, 4H, 2CH2), 3.75 (m, 2H, 2CH), 4.60 (d, J=10 Hz, 2H, 2CH), 4.65 (Bs, 2H, 2OH), 4.80 (Bs, 4H, 2NH2), 5.19 (s, 2H, 2CH), 5.83 (t, J=15 Hz, 2H, 2CH═), 5.89 (d, J=10 Hz, 2H, 2CH═), 6.58 (t, J=15 Hz, 2H, 2CH═), 6.94 (d, J=10 Hz, 2H, 2CH═), 7.17 (m, 2H, 2NH), 7.24 (s, 2H, 2CH═), 9.20 (s, 2H, 2NH); MS (m/z)1189 (M+H).

The corresponding HCl salt was prepared by the following method: an HCl solution in EtOH (5 ml, 0.123N) was added to a solution of compound #237 (1 gm as prepared above) in THF (15 ml) and EtOH (50 ml) at room temperature. The reaction mixture was stirred for 10 min. The salt was precipitated, filtered and washed with large amount of EtOH and dried in vacuo.

### EXAMPLE 7

### Lyophilization

Lyophilization of the emulsions from Examples 5 and 6 is accomplished according to one or more of the schemes contained in the following Table.

| Initial | Final | | |
|---|---|---|---|
| Temp. | Temp. | Pressure | |
| (° C.) | (° C.) | (mTorr) | Action |
| 25 | −40 | Ambient | Ramp at 1° C./min |
| −40 | −40 | Ambient | Hold for 60 min |
| −40 | −40 | 50 | Condenser at |
| | | | −60° C. to −80° C. |
| −40 | −28 | 50 | Ramp at 1° C./min |
| −28 | −28 | 50 | Hold for 7200 min |
| −28 | 30 | 50 | Ramp at 1° C./min |
| 30 | 30 | 50 | Hold for 300 min |

Complete
Stopper vials under N₂ at approximately 0.9 atm

One of ordinary skill will appreciate that the parameters described in the preceding table can be adjusted depending on the conditions used, and depending on whether and to what extent the methods of formulation and amounts of materials used are scaled up or down, or varied, with respect to one another.

The foregoing examples are not limiting and merely representative of various aspects and embodiments of the present invention. All documents cited are indicative of the levels of skill in the art to which the invention pertains. The disclosure of each document cited is incorporated by reference herein to the same extent as if each had been incorporated by reference in its entirety individually, although none of the documents is admitted to be prior art.

One skilled in the art will readily appreciate that the present invention is well adapted to carry out the objects and obtain the ends and advantages mentioned, as well as those inherent therein. The methods and compositions described illustrate preferred embodiments, are exemplary, and are not intended as limitations on the scope of the invention. Certain modifications and other uses will occur to those skilled in the art, and are encompassed within the spirit of the invention, as defined by the scope of the claims.

The reagents described herein are either commercially available, e.g., from Sigma-Aldrich, or else readily producible without undue experimentation using routine procedures known to those of ordinary skill in the art.

It will be readily apparent to one skilled in the art that varying substitutions and modifications may be made to the invention without departing from the scope and spirit of the invention. Thus, such additional embodiments are within the scope of the invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced With either of the other two terms, and each has a different meaning within the patent laws. The terms and expressions which have been employed are used as terms of description and not of limitation, and there is no intention in the use of such terms and expressions of excluding any equivalents of the features shown and described, or portions thereof. It is recognized that various modifications are possible within the scope of the invention claimed. Thus, it should be understood that although the present invention has been specifically disclosed by preferred embodiments, optional features, modifications and variations of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group, and exclusions of individual members as appropriate.

Other embodiments are within the following claims.

1. A method of preparing an ansamycin emulsion comprising:
(a) providing an ansamycin or pharmaceutically acceptable salt thereof dissolved in an ethanol solution;
(b) mixing said ansamycin or pharmaceutically acceptable salt thereof with a medium chain triglyceride to form a first mixture;
(c) substantially removing said ethanol from said first mixture if it is present;
(d) combining the product of step (c) with an emulsifying agent and a stabilizer to form a second mixture;
(e) emulsifying said second mixture;
(f) optionally lyophilizing the product of step (e); and
(g) optionally hydrating the product of step (f).

2. The method of claim 1 wherein said ansamycin or pharmaceutically acceptable salt thereof is selected from a member selected from the group of geldanamycin, pharmaceutically acceptable salts of geldanamycin, geldanamycin derivatives and pharmaceutically acceptable salts of geldanamycin derivatives.

3. The method of claim 1 wherein said ansamycin or pharmaceutically acceptable salt thereof is selected from the group consisting of 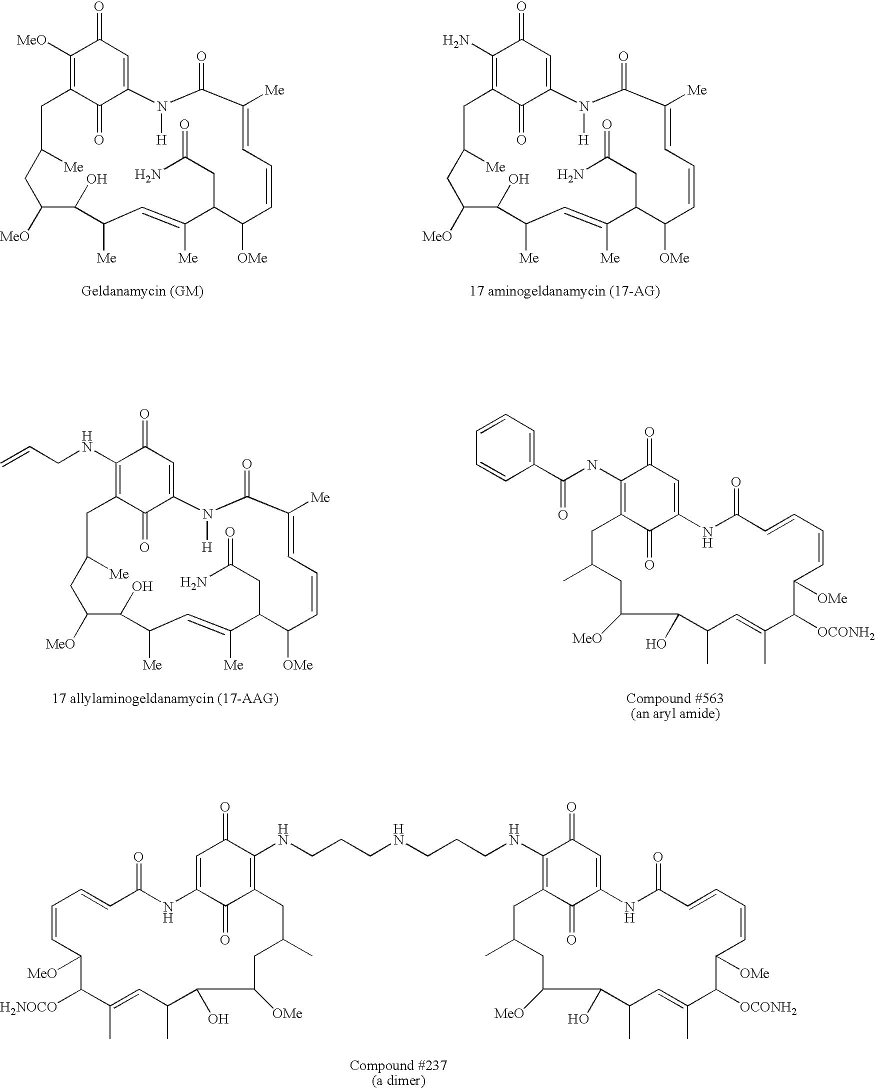
and pharmaceutically acceptable salts thereof.

4. The method of claim 1 wherein said medium chain triglyceride comprises one or more of caprylic acid and capric acid, wherein said emulsifying agent comprises a phosphotidylcholine, and wherein said stabilizer comprises sucrose.

5. The method of claim 1 wherein said emulsified second mixture has an average oil droplet size of about 400 nm or less.

6. The method of claim 1 wherein said second mixture is filter-sterilized.

7. The method of claim 1 wherein said drug in said emulsified second mixture is present at a concentration of about 3 mg/ml or less.

8. The method of claim 1 wherein said emulsifying step comprises using one or more members selected from the group consisting of mechanical mixing, ultrasonic irradiation, passage through a microfluidizer, and passage across one or more membranes.

9. The method of claim 1 further comprising the step (f) of lyophilizing the product of step (e).

10. The method of claim 9 wherein the product of step (f) is subsequently hydrated by use of a step (g).

11. The method of claim 10 further comprising packaging the product of step (g) or (f) in a container.

12. The method of claim 11 wherein said container is light-resistant.

13. The method of claim 1 wherein one or more of said steps is performed under reduced lighting.

14. The method of claim 1 wherein one or more of said steps is performed under an inert gas.

15. A method of preparing an ansamycin emulsion, comprising:
(a) dissolving an ansamycin or pharmaceutically acceptable salt thereof in a preformed solution comprising an emulsifying agent dissolved in a medium chain triglyceride solution; said dissolving optionally employing the application of heat;
(b) combining the product of step (a) with a stabilizer;
(c) emulsifying the product of step (b);
(d) optionally lyophilizing the product of step (c); and
(e) optionally hydrating the product of step (d).

16. The method of claim 15 wherein said ansamycin or pharmaceutically acceptable salt thereof is selected from a member selected from the group of geldanamycin, pharmaceutically acceptable salts of geldanamycin, geldanamycin derivatives and pharmaceutically acceptable salts of geldanamycin derivatives.

17. The method of claim 15 wherein said ansamycin or pharmaceutically acceptable salt thereof is selected from the group consisting of 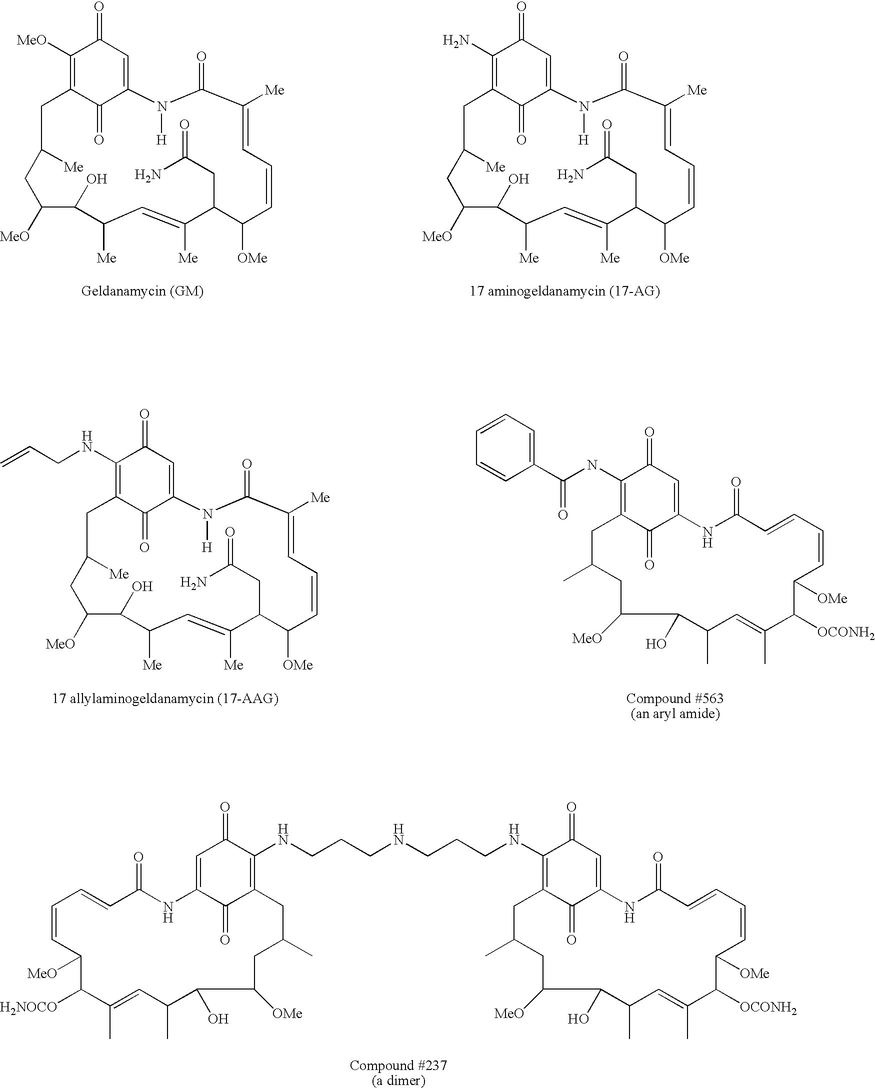
and pharmaceutically acceptable salts thereof.

18. The method of claim 15 wherein said medium chain triglyceride comprises one or more of caprylic acid and capric acid, wherein said emulsifying agent comprises soy phospholecithin, and wherein said stabilizer comprises sucrose.

19. The method of claim 15 wherein said emulsified second mixture has a mean oil droplet size of about 400 nm or less.

20. The method of claim 14 wherein said drug in said emulsified second mixture is present at a concentration of about 2 mg/ml or less.

21. The method of claim 15 wherein said emulsifying step (c) comprises using one or more members selected from the group consisting of mechanical mixing, ultrasonic irradiation, passage through a microfluidizer, and passage across one or more membranes.

22. The method of claim 15 wherein one or more of said steps is carried out under inert atmospheric conditions.

23. An emulsion prepared using any one of the methods of claims 1-22.

24. An emulsion comprising an oil phase and an aqueous phase, wherein said oil phase comprises an ansamycin or pharmaceutically acceptable salt thereof, a medium chain triglyceride, and a lecithin, wherein said aqueous phase comprises one or more stabilizers, and wherein said emulsion is optionally one or more of lyophilized and frozen.

25. The emulsion of claim 24 wherein said ansamycin or pharmaceutically acceptable salt thereof is selected from a member selected from the group of geldanamycin, pharmaceutically acceptable salts of geldanamycin, geldanamycin derivatives and pharmaceutically acceptable salts of geldanamycin derivatives.

26. The emulsion of claim 24 wherein said ansamycin is or pharmaceutically acceptable salt thereof is selected from the group consisting of 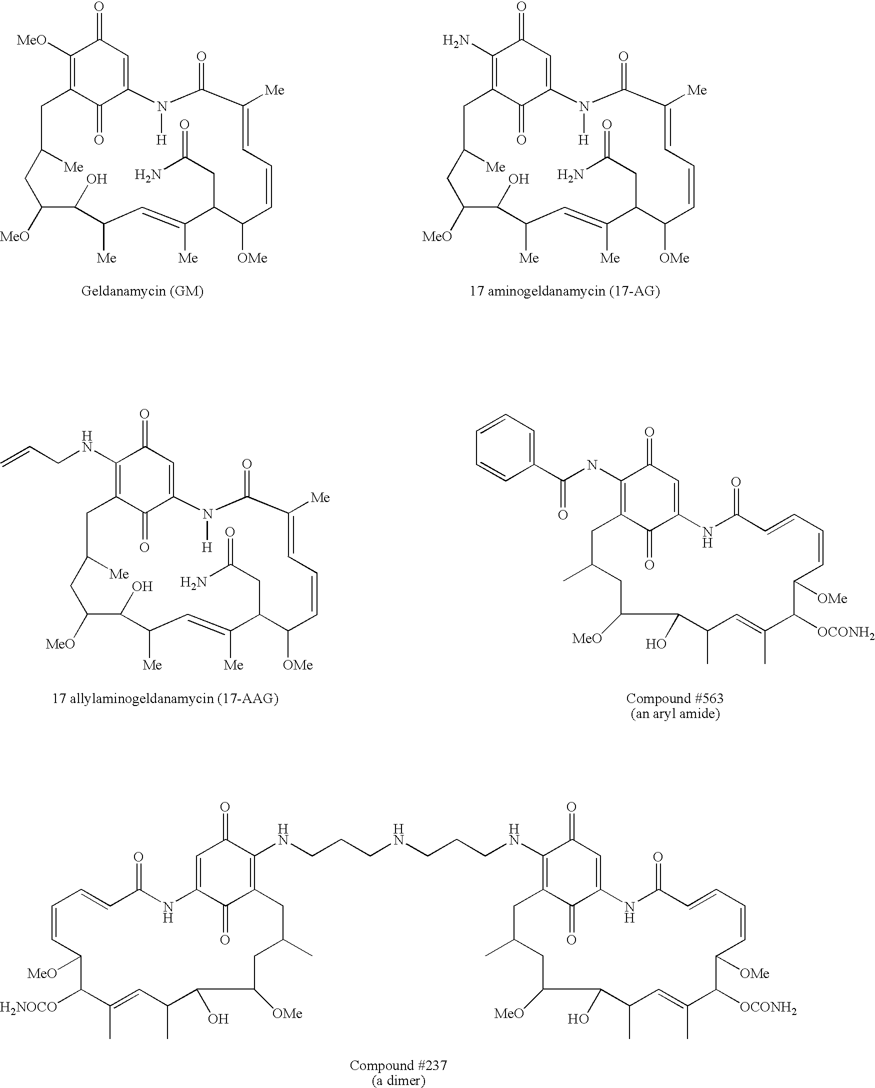
and pharmaceutically acceptable salts thereof.

27. The emulsion of claim 24 wherein said ansamycin or pharmaceutically acceptable salt thereof is soluble in said oil phase in a concentration range of between about 5-30 mg/ml inclusive.

28. The emulsion of claim 24 wherein said ansamycin or pharmaceutically acceptable salt thereof is present at a concentration of up to about 3 mg/ml in said emulsion.

29. The emulsion of claim 24 characterized by a mean droplet size of about 400 nm or less.

30. A method of treating or preventing a disorder in a mammal, comprising:
administering to a mammalian subject a pharmaceutically effective amount of the emulsion produced according to claim 24.

31. A method of treating or preventing a disorder in a mammal, comprising:
administering to a mammalian subject a pharmaceutically effective amount of an emulsion according to any one of claims 24-29.

32. The method of claim 30 wherein said disorder is selected from the group of disorders consisting of ischemia, proliferative disorders, infections, neurological disorders, tumors, leukemias, neoplasms, cancers, carcinomas, and malignant diseases.

33. The method of claim 31 wherein said proliferative disorder is selected from the group consisting of tumors, inflammatory diseases, fungal infection, yeast infection, and viral infection.

34. The method of claim 1 wherein said emulsified second mixture has an average oil droplet size of about 200 nm or less.

35. The method of claim 15 wherein said emulsified second mixture has an average oil droplet size of about 200 nm or less.

36. The emulsion of claim 24 characterized by an average oil droplet size of about 200 nm or less.

37. The method of claim 1, 15, 30, or 31 wherein said ansamycin is 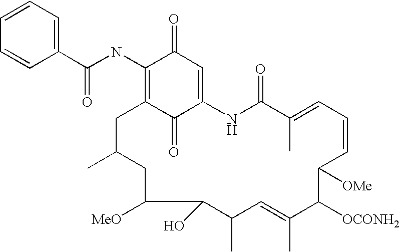
or a pharmaceutically acceptable salt thereof.

38. The method of claim 1, 15, 30, or 31 wherein said ansamycin is 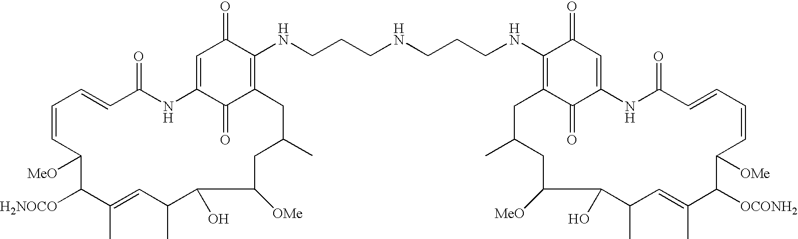
or a pharmaceutically acceptable salt thereof.

39. The emulsion of claim 24 wherein said ansamycin is 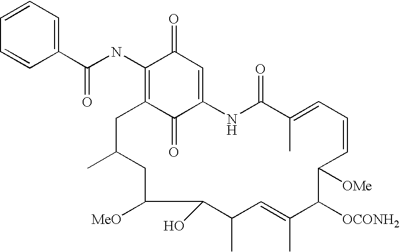
or a pharmaceutically acceptable salt thereof.

40. The emulsion of claim 24 wherein said ansamycin is 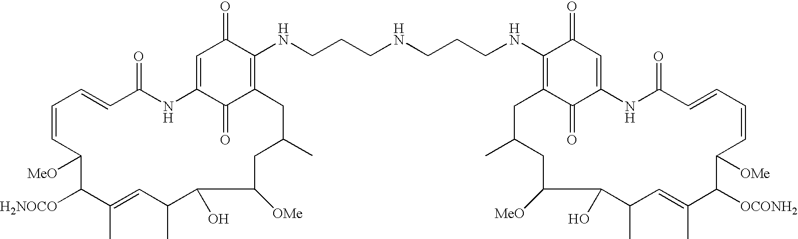
or a pharmaceutically acceptable salt thereof.

41. The method of claim 38 wherein said emulsion comprises mannitol, one or more buffers and, optionally, dextrose.

42. The method of claim 41 wherein said emulsion comprises about 1-1.5% (w/v) ansamycin, about 5% (w/v) mannitol, and about 10-20 mM sodium acetate (pH˜5).

43. The method of claim 38 wherein said emulsion comprises between about 1 and 10 mg/ml inclusive of said ansamycin.

44. The emulsion of claim 40 wherein prior to said optional lyophillization, said emulsion comprises mannitol, one or more buffers and, optionally, dextrose.

45. The emulsion of claim 44 comprising about 1-1.5% (w/v) ansamycin, about 5% (w/v) mannitol, and about 10-20 mM sodium acetate (pH˜5).

46. The emulsion of claim 40 comprising between about 1 and 10 mg/ml inclusive of said ansamycin.
